(19) United States
(12) Patent Application Publication
(10) Pub. No.: US 2026/0267729 A1
(43) Pub. Date: Sep. 10, 2026

(54) ADAPTIVE AND DYNAMIC HOSTING OF DATA-DRIVEN DIAGNOSTIC CONTENT BY CONTINUOUS MONITORING AND EVALUATION FOR PROACTIVE MAINTENANCE SERVICES

(71) Applicant: KONINKLIJKE PHILIPS N.V., EINDHOVEN (NL)

(72) Inventors: Sauvik BHATTACHARYA, EINDHOVEN (NL); Tiblets Zeray DEMEWEZ, EINDHOVEN (NL); Mauro BARBIERI, EINDHOVEN (NL)

(21) Appl. No.: 19/491,421

(22) PCT Filed: Jun. 19, 2024
(86) PCT No.: PCT/EP2024/067006

§ 371 Date: Dec. 9, 2025

Related U.S. Application Data
(60) Provisional application No. 63/523,383, filed on Jun. 27, 2023.

(30) Foreign Application Priority Data
Jul. 11, 2023 (EP) 23184718.7

(51) Int. Cl. G06F 11/07; G16H 40/67

(52) U.S. Cl. CPC G06F 11/079; G16H 40/67

(57) ABSTRACT

A distributed computing network includes an edge computing device and a server computer. The edge computing device receives log data generated by the associated medical device, and is programmed to: perform monitoring of the medical device by analysis of the log data received by the edge computing device; and output alerts generated by the monitoring performed at the edge computing device. The server computer receives the log data from the edge computing device via the Internet, and is programmed to: perform the monitoring of the associated medical device by analysis of the log data received at the server computer; and output alerts generated by the monitoring performed at the server computer. The distributed computing network performs the monitoring of the medical device over time by, at a given time, selecting one of the computing device or the server computer to perform the monitoring of the medical device.

### FIELD OF THE INVENTION

The following relates generally to the medical device maintenance arts, medical imaging device maintenance arts, medical device log file analysis arts, medical predictive maintenance arts, and related arts.

### BACKGROUND OF THE INVENTION

Multiple medical imaging systems are typically located at a hospital or site. These systems are communicatively connected to a common electronic network, such as a hospital data network. The medical imaging systems that are located at the same site share the communication bandwidth of a communication channel between the site and the central data processing and storage system. An edge device, comprising a computer servicing the hospital or a portion thereof, e.g., the hospital's radiology department, may collect data from the medical imaging devices at that hospital and then forward the collected data to the central data processing and storage system. This conveniently provides uniformity as the edge devices servicing various hospitals can be standardized, for example provided by the medical imaging devices vendor, so that the central data processing and storage system does not need individualized interfaces for connecting with a wide range of different hospital data networks. A given edge device may be implemented as a physical server or as a virtual machine (VM) running on a server.

Vendors of medical device systems can provide proactive and predictive maintenance services to their customers. More generally, such diagnostic models can generate alerts predicting likely failure events (optionally with a timeframe), and/or can generate alerts about existing problems detected by the models. These services rely on data that comes from medical systems on a regular basis. For this, connectivity of the medical systems is crucial. Moreover, a reliable analytics platform that collects, processes and stores data to build diagnostic models or applications is essential. Typically, these processes take place on the cloud or on-premises, i.e., at the location hosting the medical device.

In a typical arrangement, log data automatically generated by medical imaging devices is uploaded to a server or cloud computing resource maintained by the vendor or other maintenance service provider, and the diagnostic models are also deployed at the server or cloud computing resource. This has advantages such as facilitating updating of the models at a central point, and facilitating communication of alerts to service engineers employed by the vendor or other service provider. However, server or cloud-based models rely heavily on the connectivity of the medical systems, availability, and correctness of the end-to-end data pipeline as well as model deployment infrastructure. Connectivity of medical systems can be hampered by incorrect registration of systems, network interruptions at a customer or vendor site, etc. Furthermore, data pipelines should be robust and reliable to ensure uninterrupted flow of data. Interruptions in data flow disable the proactive services fueled by them, leading possibly to system downtime. Such problems can be remediated to some extent by improving the utilized Internet infrastructure, but the quality of Internet connectivity may be outside the control of both the customer (e.g., hospital) and the vendor or other service provider.

The following discloses certain improvements to overcome these problems and others.

### SUMMARY OF THE INVENTION

In one aspect, a system is disclosed for monitoring a medical device. The system includes a distributed computing network comprising an edge computing device and a server computer. The edge computing device is operatively connected to receive log data generated by the associated medical device, and is programmed to: perform monitoring of the medical device by analysis of the log data received by the edge computing device; and output alerts generated by the monitoring performed at the edge computing device. The server computer is configured to receive the log data from the edge computing device via the Internet, and is programmed to: perform the monitoring of the associated medical device by analysis of the log data received at the server computer; and output alerts generated by the monitoring performed at the server computer. The distributed computing network is configured to perform the monitoring of the medical device over time by, at a given time, selecting one of the edge computing device or the server computer to perform the monitoring of the medical device.

In another aspect, at least one non-transitory computer readable medium stores one or more diagnostic models configured to analyze log data generated by a medical device, and instructions readable and executable by at least an edge computing device. The instructions are executable to: monitor the medical device by applying the one or more diagnostic models to generate alerts indicative of problems with the medical device; analyze a plurality of factors to determine whether the monitoring of the medical device should be transferred from the edge computing device to a vendor server; and transfer the monitoring of the medical device from the edge computing device to the vendor server based on the analysis determining that the monitoring of the medical device should be transferred from the edge computing device to the vendor server.

In another aspect, a distributed computing method is disclosed, including: monitoring a medical device by applying one or more diagnostic models to generate alerts indicative of problems with the medical device; analyzing a plurality of factors to determine whether the monitoring of the medical device should be transferred from an edge computing device (14) to a vendor server; and generating an alert indicating that the monitoring of the medical device should be transferred from the edge computing device to the vendor server based on the analysis determining that the monitoring of the medical device should be transferred from the edge computing device to the vendor server.

One advantage resides in leveraging edge computing to ensure uninterrupted availability of analytic applications for monitoring the health of medical imaging devices.

Another advantage resides in dynamically transferring data analytic applications to the customer premises or infrastructure when conditions for hosting them on a centralized (or cloud) platform are not met.

Another advantage resides in dynamically transferring a subset of data analytic applications to the customer premises or infrastructure.

Another advantage resides in dynamically transferring data analytic applications back to the centralized (i.e., cloud) platform when conditions for central hosting are again met.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system analyzing log data in accordance with the present disclosure.

FIG. 2 shows exemplary flow chart operations of the system of FIG. 1.

FIG. 3 shows another embodiment of the system of FIG. 1.

### DETAILED DESCRIPTION OF EMBODIMENTS

Remote monitoring of a fleet of imaging devices is performed by a cloud server maintained by the vendor (or other imaging device service provider) which applies diagnostic models to imaging device log data uploaded to the vendor's cloud server to detect existing problems with imaging devices and/or to predict likely failures in support of proactive maintenance. While this arrangement usually works well, it relies on continuous Internet connectivity between the cloud server and the hospital (or other imaging device site). However, interruptions can arise due to loss of Internet connectivity between a given hospital and the cloud server handling the remote monitoring. Such an interruption, if sufficiently extended, can result in missing important predictive maintenance alerts.

In some installations an edge computing device is maintained locally at the hospital to improve interfacing between the hospital and the vendor's cloud server. In this arrangement, the log data is transferred to the edge computing device which then transfers the log data to the cloud server. The edge computing device may be a dedicated physical server computer or may be implemented as a virtual machine (VM) on the hospital's information technology (IT) server computer. This has advantages, such as providing for data latency in transfer of the log data (e.g., the data can be buffered at the edge computing device before being transferred to the cloud server), and simplifying the interfacing as the edge computing device provides a consistent (e.g. same) interface with the cloud server across the fleet of imaging devices.

The disclosed system leverages the edge computing device to also provide a temporary backup capable of performing the remote monitoring locally on the edge computing device at times when connectivity with the cloud server is lost or deemed insufficiently reliable. One or more factors can be analyzed to determine when it would be advantageous or necessary to transfer the remote monitoring from the vendor's cloud server to the local edge computing device. This analysis is done in the cloud and/or at the edge computing device. Advantageously, the edge computing device is already configured to receive the log data from the imaging devices at the hospital or other customer served by the edge device, and so that data is available for analysis at the edge computing device.

There are significant benefits to primarily hosting the diagnostic analyses at the vendor-maintained cloud-based server. The edge computing device generally has less computing power than a cloud server, and hence the edge computing device may in some instances be unable to implement the full breadth of diagnostic models that can be hosted at the vendor's cloud server. Furthermore, the alerts generated by the diagnostic analyses are generally intended for action service engineers with the expertise for interpreting the alerts and performing any requisite maintenance. Still further, hosting the diagnostic analyses at the vendor's server facilitates centralized updates to the diagnostic models.

Hence, while in disclosed approaches the edge computing device provides a backup for performing the diagnostic analyses, this functionality is advantageously transferred back to the vendor cloud server when reliable Internet connectivity is restored. In some embodiments, the analysis to determine when the monitoring should be transferred back to the cloud server can be performed at the local edge computing device or at the cloud server, or by a joint decision such as having the local server send a notice to the cloud indicating it is ready to hand the remote monitoring back to the vendor's cloud server and then having the cloud server make the final decision.

To implement the approach, a copy of the remote monitoring software (e.g., the predictive models) is installed at the local edge computing device. This may be difficult to do in a just-in-time (JIT) paradigm as the transfer of remote monitoring may be in response to a loss of Internet connectivity, thus making the JIT installation from the cloud difficult or impossible. Hence, the remote monitoring software in some embodiments is installed together with other software of the imaging device(s) being monitored, if the edge computing device has sufficient memory. Updates to the remote monitoring software are then pushed along with updates to the imaging device software to keep the remote monitoring capability at the local edge computing device up to date. It should be noted that the copy of the remote monitoring software (e.g., diagnostic models) that is installed at the local edge computing device may be a subset or a copy of other reduced-functionality of the remote monitoring software deployed at the vendor's cloud server. For example, a subset or a copy of reduced-functionality may be designed for execution on the edge device which has more limited processing power compared with the vendor's cloud server.

In some embodiments, one or more alerts can be generated at the local edge computing device. If the alert is of a type that can be handled by a local biomed and the hospital has such a suitably trained biomed on staff, then the alert could be handled locally, so that the vendor is never involved. If the alert cannot be handled locally then it is forwarded to the vendor (via the Internet or telephonically if there is no Internet connectivity). In this case the alert should be given high priority at the vendor since the issue is known to the customer. (Also, even if the alert is handled at the hospital, the alert along with a summary of the action taken locally at the hospital may still be forwarded to the vendor so that the vendor can update its maintenance records for the medical device appropriately).

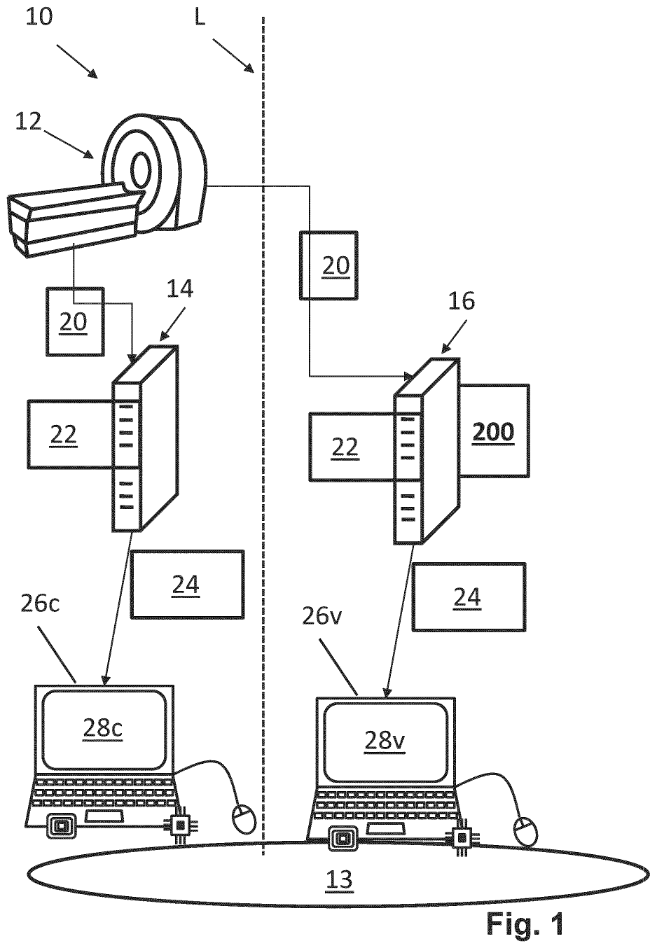

With reference to FIG. 1, an illustrative servicing support system 10 for supporting a service engineer in servicing a device 12 (e.g., a medical imaging device as shown in FIG. 1—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device or machine that automatically generates log data that are analyzed by predictive models to predict component failures, e.g., the approach could be applied to a commercial airliner, radiation therapy device, a cardo ship, an industrial robot, or so forth). In addition, although only a single device 12 is shown in FIG. 1, the support system 10 can analyze log data from a fleet of devices 12 (i.e., more than one device).

As shown in FIG. 1, the servicing support system 100 includes, or is accessible by, a distributed computing network comprising one or more distributed computers electronically connected to each other via an electronic network 13 (e.g., a wired connection, a local area network (LAN), the Internet, wireless Wi-Fi or 4G/5G interface or the like for connection to the Internet and/or an intranet, and so forth).

The distributed computing network includes one or more local customer computers 14 (i.e., “edge devices”) configured to analyze log data from the device 12. A number of edge devices 14 corresponds to the number of devices 12 in the fleet. For example, the edge device(s) 14 can be disposed adjacent to, or in a same building as, the corresponding device(s) 12. A single edge device computer 14 is shown in FIG. 1, and can comprise a server computer (e.g., single server computer, or a server cluster, or a cloud computing resource comprising ad hoc-interconnected server computers, or so forth). The edge computing device 14 could be a virtual machine (VM) running on a physical hospital server computer or the like. Whether physical or a VM, the term edge computing device 14 or edge device 14 is used herein. The distributed computing network also includes a vendor server computer 16. Each edge computing device 14 is programmed to receive log data automatically generated by the imaging device 12 and to transfer the log data to the vendor server computer 16. The vendor's server computer 16 can comprise a server computer as shown, or can be a server cluster, or a cloud computing resource comprising ad hoc-interconnected server computers. Whether implemented as single server computer, a cluster of server computers, or a cloud computing resource, the term server computer 16 is used herein to encompass all such embodiments. The distributed computing network 14, 16 thus comprises the edge computing device 14 and the server computer 16.

The medical device(s) 12 is configured to generate a dataset of log data 20 for analysis by the edge computing device 14 and the vendor server computer 16. For example, the log data 20 may include diverse information generated by sensors of the illustrative medical imaging device 12 (e.g., temperature readings for various components and so forth), fault indicators produced by components of the imaging device 12, information on imaging scans run by the imaging device 12 and other usage data, and/or so forth. The log data 20 can comprise (or at least be capable of being represented as) log data strings generated by the device 12, although other data formats are contemplated. The log data 20 for a particular device 12 can be transferred to the corresponding edge computing device 14, which then transfers the log data 20 to the vendor's sever computer 16. In addition, as shown in FIG. 1, each of the computers 14, 16 can be located in geographically diverse locations (as indicated by the dashed lines L in FIG. 1), typically with the edge computing device 14 located at the hospital or other medical facility hosting the medical device 12 and the server computer 16 being located remotely from the hospital, e.g. at the vendor's site if it is self-hosted or at a server farm if the server computer 16 is implemented as a cloud computing resource hosted by a third-party commercial provider.

Each of the edge computing device 14 and the vendor's server computer 16 also comprise a non-transitory computer readable medium configured to store one or more diagnostic models 22 configured to analyze the log data 20. The diagnostic models 22 can provide various types of diagnoses. For example, the diagnostic models 22 can include predictive models that predict a likely failure (typically with a timeframe to fail) of a component of the imaging device 12. As a nonlimiting illustrative example, in the case of a CT scanner a predictive model can predict the remaining life of the X-ray tube based on logged usage hours and/or other information such as tube current and voltage measurements. Alerts generated by a predictive model can be used for timely scheduling of preventative maintenance, such as replacing an X-ray tube before it actually fails. As another example, the diagnostic models 22 can include detection models that detect existing problems with the imaging device 12. For example, a model can detect a component that is running hot, i.e., at too-high temperature, as measured by a temperature sensor of the component. Based on such an alert, a service engineer can schedule an inspection or, if the problem is sufficiently serious, contact the hospital to recommend stopping usage of the medical device 12 until the problem is fixed. These are merely some nonlimiting illustrative examples of suitable diagnostic models 22.

For example, the vendor server computer(s) 16 are configured to also receive the log data 20 from the device(s) 12, perform monitoring of the medical device 12 by analysis of the log data 20, and output one or more alerts 24 generated by the monitoring. For example, the edge computing device(s) 14 are in communication with a corresponding vendor electronic processing device 26v (e.g., a computer, a workstation, a smartphone, a tablet, and so forth) operable by an employee of the vendor (e.g., a remote service engineer (RSE), a field service engineer (FSE), and so forth). A graphical user interface (GUI) 28v is displayed on a display device of the electronic processing device 26v to display the alert(s) 24. The alert(s) 24 can be indicative of a performance of the device 12 based on the analysis of the log data 20.

In addition, the edge computing device(s) 14 are configured to receive the log data 20 from the device(s) 12, perform monitoring of the medical device 12 by analysis of the log data 20, and output one or more alerts 24 generated by the monitoring. For example, the edge computing device(s) 14 are in communication with a corresponding local customer electronic processing device 26c (e.g., a computer, a workstation, a smartphone, a tablet, and so forth) operable by an employee of the customer (e.g., a technician and so forth). A local graphical user interface (GUI) 28c is displayed on a display device of the electronic processing device 26c to display the alert(s) 24. The alert(s) 24 can be indicative of a performance of the device 12 based on the analysis of the log data 20. In some examples, the edge computing device 14 is further programmed to classify each alert 24 as to whether the alert 24 requires remote assistance, and to additionally output alerts 24 classified as requiring remote assistance to the vendor server computer 16.

The distributed computing network 14, 16 is configured to perform the monitoring of the medical device 12 over time by, at a given time, selecting one of the edge computing device 14 or the server computer 16 to perform the monitoring of the medical device 12. In one example, this selection is based on at least on a quality metric of Internet connectivity between the server computer 16 and the edge computing device 14 at the given time. In another example, this selection is based on an indication generated by the edge computing device 14 of whether the edge computing device 14 is ready to perform the monitoring of the medical device 12. In another example, if the edge device 14 abruptly loses all connectivity with the server computer 16 then the edge device 14 can select itself (i.e., select the edge device 14) to perform the monitoring until its connection with the server computer 16 is re-established.

At some point in time, the server computer 16 is configured to transfer software executable to perform the analysis of the log data 20 received by the edge computing device 14 from the server computer 16 to the edge computing device 14. This could in some instances be done at the time the selection is made to have the edge computing device 14 temporarily take over the monitoring. However, in practice this may be impractical, since the decision to transfer monitoring may be made in response to detection of unreliable connectivity between the edge device 14 and the server computer 16 (at which point the unreliable connection may preclude transferring the software). Hence, in some embodiments the software transfer is done at an earlier time, such as when the edge computing device 14 is installed. To transfer the software from the server computer 16 to the edge computing device 14, the software is optionally encrypted at the server computer 16 before transfer. The encrypted software is transferred from the server computer 16 to the edge computing device 14, and the encrypted software is decrypted and deployed at the edge computing device 14.

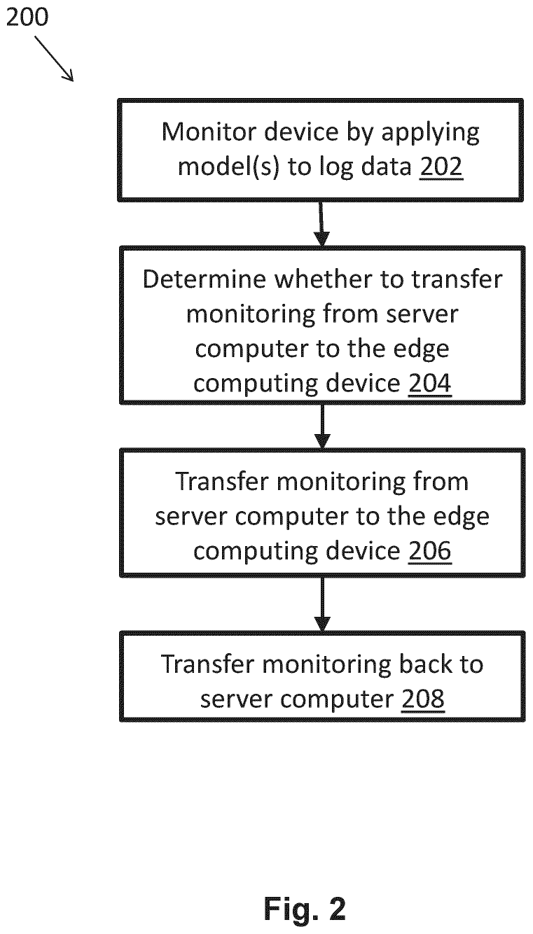

With continuing reference to FIG. 1 and further reference to FIG. 2, an illustrative embodiment of the method 200 is diagrammatically shown as a flowchart. In some examples, the method 200 may be performed at least in part by cloud processing.

To begin the method 200, at an operation 202, the server computer 16 is configured to monitor the medical device 12 by applying the diagnostic model(s) 22 to the log data 20 received at the server computer 16 from the medical device 12 via the edge device 14 to generate the alerts 24 indicative of problems with the medical device 12. These alerts will generally be displayed at the vendor's GUI 26v (see FIG. 1) for review and possible action by a service engineer or other agent of the vendor.

At an operation 204, the distributed computing network 14, 16 is configured to analyze a plurality of factors to determine whether the monitoring of the medical device 12 should be transferred from the server computer 16 to the edge computing device 14. The factors can include, for example, one or more of a status of connectivity between the edge device 14 and server computer 16, a presence of suitable infrastructure (e.g. the edge computing device 14 with a copy of the diagnostic models 22) at the edge device 14, a number of technicians at the hospital or other site of the edge computing device 14, and so forth. As previously mentioned, the selection operation 204 is performed by the distributed computing network 14, 16, and may in a given embodiment be performed entirely by the server computer 16, or entirely by the edge device 14, or may be performed cooperatively by both devices 14 and 16 (e.g., the server computer 16 may send a request to take over monitoring to the edge device 14 and the edge device 14 then accepts the request to complete the selection of the edge device 14). The selection 204 may also in some embodiments be performed at a given time by different devices, e.g. normally the server computer 16 may make the selection 204 but if there is an abrupt and complete loss of Internet connectivity then the selection may be made by the (now isolated) edge computing device 14 on its own. The factors for making the selection 204 can also include an availability of the log data 20 and a presence of historical log data for the medical device 12.

At an operation 206, the distributed computing network 14, 16 is configured to transfer the monitoring of the medical device 12 from the server computer 16 to the edge computing device 14 based on the analysis operation 204 determining that the monitoring of the medical device 12 should be

After such a transfer, the distributed computing network 14, 16 continues to analyze the factors to determine when the server computer 16 should resume the monitoring of the medical device 12. At an operation 208, the monitoring of the medical device 12 is transferred from the edge computing device 14 back to the server computer 16. In some examples, the server computer 16 can generate and transmit a notification from the server computer 16 to the edge computing device 14 indicating readiness of the server computer 16 to resume the monitoring.

### EXAMPLES

The system 10 is configured to address the dependency of proactive and predictive maintenance services on uninterrupted connectivity and data availability by using edge computing. Edge computing is a technique that enables to deploy models on a device or in the vicinity of a device. The system 10 is configured to regularly assess a cloud deployment platform of diagnostic models 22 using dynamic measures that assist in deciding whether to keep the model 22 in the cloud or temporarily move them to the edge computer 14. This ensures continuity of providing proactive and predictive services. When the situation at hand improves, the model 22 is moved back to the vendor server 16 to take advantage of centralized hosting on the cloud.

The dynamic measures are defined based on the characteristics of the customer and the content of the model as described below. These measures can include, for example, a status of the connectivity of the device 12 at the customer's site, an IT infrastructure to enable alerting to the hospital personnel, a biomed setup (i.e., “Does the customer have biomeds that can follow up when an alert is generated by a diagnostic model at the customer's site?”), and so forth. The models 22 can include a dependency on immediate data availability (i.e., “Does the model need real-time data, or can it deal with a latency of e.g. a few days?”), and a dependency on historical data (i.e., “Does the model need to use a (large) set of historical data, or can it make a prediction based on relatively little data?”).

In case of edge-based model, a biomed gets notification whenever an alert 24 is generated. The biomed reports to the vendor that assign the right personnel to resolve the issue. The vendor pays a fee to the customer for every alert reported by a biomed. Cloud-based models are managed in the existing process where an alert is reported directly to vendor personnel.

Leveraging edge computing for maintenance services offers the possibility of applying proactive and predictive maintenance services to a wider set of devices including the ones that have connectivity issue due to poor internet connection. Consequently, it reduces data transfer, processing, and storage costs. Moreover, it minimizes onsite diagnostic cost and avoids planned maintenance services. It also allows a vendor to offer such services to smaller hospitals who do not have the necessary IT and network infrastructure in place to ensure uninterrupted flow of data to the central (vendor) cloud. It also allows extending such services to hospitals that do not wish to, or are restricted by regulation or policy (e.g., military hospitals), connect their devices to the Internet.

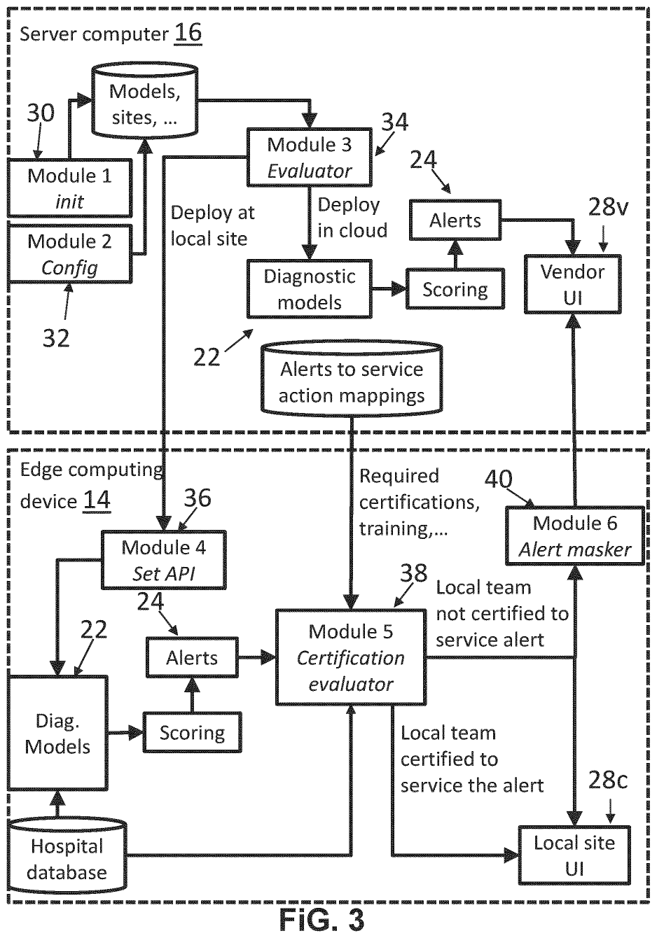

FIG. 3 shows another embodiment of the system 10. FIG. 3 shows a detailed overview of the various modules and data sources involved in the system 10. It consists of multiple parameters, as well as functional modules that use these parameters as input to execute the invention. Let M={m₁, m₂, . . . , mᵢ}, a set of i proactive and predictive 22, and let S={s₁, s₂, . . . , sⱼ}, a set of j sites hosting (medical) systems (e.g., a number of edge device computers 14) which are in scope of the set of models M 22 apply.

The top-level computer 18 is configured to analyze a function C={c, u, v, t)₁, (c, u, v, t)₂, . . . , (c, u, v, t)ₖ}, a set of k quadruples defined per site s∈S, capturing characteristics of the site (i.e., the edge computer 14) and whether it is suitable for hosting the models 22. c is a characteristic or metric that is evaluated when deciding whether to host m in the vendor server 16 or transfer it to the local infrastructure of the edge computer 14 at s. u is the last known value of the characteristic c for the site s. v is the ideal value of the characteristic c recorded over all sites in the set S. t is a noise tolerance factor that allows to ignore minor fluctuations in the value of u, when comparing it against the ideal value v. In a first example, a status of the connectivity of the device at a specific customer site is analyzed, in which c is an “Average log file download rate for systems at site”, u=75%, v=98%, t=1 representing that there is a 1% chance of the value of u being an outlier or too noisy to be compared with v, making a resampling necessary. In a second example, a suitability of local IT infrastructure for hosting models 22 is analyzed, in which c is a “Number of multi-threading workstations with more than 15 GB of RAM”, u=2, v=1, and t=N/A. In a third example, a determination is made as to “Does the customer have biomedical engineers that can follow up on alerts 24 generated by the models 22?”, in which c is a “Number of biomedical engineers locally available per monitored system”, u=1, v=2, t=N/A.

Let [symbol] ={(μ, p)₁, (μ, p)₂, . . . , (μ, p)ₗ}, a set of I tuples for each model m∈M, capturing characteristics of the model 22. μ is a dependency or need of the model 22, (e.g., “Longest tolerable time difference in days between current date and date of the latest available data for the model to evaluate” (real time data needed? or “Lowest tolerable time difference in days between current date and date of the oldest available data” (how much historical data is needed to reinforce model prediction?), and so forth). p is the value for each μ. Exemplary values corresponding to the above examples of μ could be “1”, “365” etc.

FIG. 3 also shows different phases of the method 200. In an initialization phase, a Module 1 “Init” 30 allows a user to define and initialize values of all parameters, including models 22, sites, characteristics etc. This module 30 also allows adding new models 22, sites, or characteristics during operation. A Module 2 “Config” 32 allows a user to define how frequently the invention evaluates where to host models 22. This evaluation is done for all models 22 in M. For example, setting this to 1 day, 12:00 AM ensures that every midnight the site and model characteristics C, [symbol] for all sites s∈S and all models m∈M are evaluated using the latest known data to decide if the model 22 should continue to be hosted in the vendor server 16, or be offloaded to the site of the edge computer 14.

In a decision phase, a module 3 “Evaluator” 34 is implemented. At a frequency and time of day configured by module 3 “Config”, this module 34 chooses whether to continue hosting a model 22 centrally in the cloud vendor server 16 to offload it to its local site of the edge computer 14, by evaluating site and model characteristics C, [symbol] for all sites s∈S and all models m∈M. It does this for all sites s∈S and all models m∈M. Note that site characteristics C capture live data as well. For every model m∈M and site s∈S, the evaluator module 34 consists of a function char(m, s)={C, [symbol] } which returns the characteristics C defined for m and s, and m's own characteristics [symbol] . The evaluator module 34 also consists of a function d(m, s, char(m, s))∈{0,1} that returns the evaluation decision of whether the model m should be offloaded to the site s (output 1) or not (output 0).

In a transfer phase, depending on the result of the decision phase 222 (via the output of the function d(m, s, char(m, s))), the model 22 in the form of its associated data analytics functions are packaged, encrypted and offloaded to the local infrastructure of the site of the edge computer 14.

In a “Preparation stage for offloaded deployment” phase, a module 4 “Set_API” 36 allows a user at a given site s∈S to define the input and output APIs for the model 22 when it is hosted locally at the edge computing device 14. This ensures that the model execution is agnostic to and integrates easily into the local infrastructure. The input API can then be used by the local infrastructure to pass required input parameters to the model 22, and the output API allows the model output to be consumed by local monitoring or response tools.

At an execution phase, a Module 5 “Certification validator” 38 is configured to check whether the local operational team of the site where the model 22 has been offloaded to (e.g., biomedical engineers at the hospital site) has required certification for direct servicing of an alert 24. This can be implemented by maintaining a mapping of model and alert categories and their corresponding service actions, to the required certifications. If the result of the check indicates that the local team is trained and certified, the full unfiltered alert 24 with all details is sent via the configured output API to the local operational team's infrastructure (e.g., a hospital dashboard). A Module 6 “Alert masker” 40 receives the output of the “certification validator” 38, and masks/removes alert details if the local operational team does not have required training. It then sends this masked alert via the configured output API 36 to the local operational team, and allows them to forward the alert with priority to the manufacturer.

In some embodiments, the “Config” module 32 allows a user to set evaluation frequencies per model 22. Thus, instead of a global frequency and time of day when all models m∈M for all sites s∈S are evaluated, the user can define a set of i evaluation frequencies for each model 22. The default evaluation frequency for any model 22 is twice its execution frequency. This ensures that the decision of whether to transfer it to a different hosting location is taken and executed well in advance of execution of the model.

In some embodiments, the maximum amount of time allowed to transfer a model to a different hosting location, e.g., offloaded from the vendor server 16 to the edge computing device 14 or vice versa, is upper bounded to a fixed default value. (The characteristics of) any new model 22 or site that is added to the sets M and S by the “Init” module to verify whether the time required for the model 22 to be offloaded to that site, or transferred to the cloud from the site within the maximum limit. This embodiment can lead to performance improvements in situations where models execute frequently, e.g., multiple times a day. Upper bounding the acceptable transfer time allows the benefits of dynamic transfer of hosting without interrupting the model execution.

In some embodiments, depending on the capabilities of the site to which a model 22 is offloaded (represented by the site characteristics parameter C={(ç, u, v, t)₁, (ç, u, v, t)₂, . . . , (ç, u, v, t)ₖ}) and the needs of the model 22 (represented by the model characteristics [symbol] ={(μ, p)₁, (μ, p)₂, . . . , (μ, p)ₗ}), only a subset of the data analytics functions critical to the functioning of the model are offloaded to the local site. For example, if the site characteristics indicate that the local infrastructure is of low specifications, then only the functions required to generate the alert 24 itself, and primary output parameters about the alert and the system may be transferred to the local site. Additional functions that generate secondary output parameters may be skipped.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system for monitoring an associated medical device, the system comprising:
a distributed computing network comprising an edge computing device and a server computer, wherein:
the edge computing device is operatively connected to receive log data generated by the associated medical device, and is programmed to:
perform monitoring of the medical device by analysis of the log data received by the edge computing device; and
output alerts generated by the monitoring performed at the edge computing device;
the server computer is configured to receive the log data from the edge computing device via the Internet, and is programmed to:
perform the monitoring of the associated medical device by analysis of the log data received at the server computer; and
output alerts generated by the monitoring performed at the server computer; and
the distributed computing network is configured to perform the monitoring of the medical device over time by, at a given time, selecting one of the edge computing device or the server computer to perform the monitoring of the medical device.

2. The system of claim 1, wherein the distributed computing network is configured to select the one of the edge computing device or the server computer to perform the monitoring of the associated medical device at a given time based at least on a quality metric of Internet connectivity between the server computer and the edge computing device at the given time.

3. The system of claim 2, wherein the distributed computing network is configured to select the one of the edge computing device or the server computer to perform the monitoring of the associated medical device at a given time further based on an indication generated by the edge computing device of whether the edge computing device is ready to perform the monitoring of the medical device.

4. The system of claim 1, wherein the distributed computing network is configured to program the edge computing device to perform the monitoring of the associated medical device by:
transferring software executable to perform the analysis of the log data received by the edge computing device from the server computer to the edge computing device.

5. The system of claim 4, wherein the transferring of the software from the server computer to the edge computing device includes:
encrypting the software at the server computer wherein the encrypted software is transferred from the server computer to the edge computing device; and
decrypting the encrypted software at the edge computing device.

6. The system of claim 1, wherein:
the server computer is further programmed to output the alerts generated by the monitoring performed at the server computer to an associated remote user interface; and
the edge computing device is further programmed to output the alerts generated by the monitoring performed at the edge computing device to an associated local user interface different from the associated remote user device.

7. The system of claim 6, wherein the edge computing device is further programmed to classify each alert generated by the monitoring performed at the edge computing device as to whether the alert requires remote assistance and to additionally output alerts classified as requiring remote assistance to the associated remote user interface.

8. At least one non-transitory computer readable medium storing:
one or more diagnostic models configured to analyze log data generated by a medical device;
instructions readable and executable by at least an edge computing device to:
monitor the medical device by applying the one or more diagnostic models to generate alerts indicative of problems with the medical device;
analyze a plurality of factors to determine whether the monitoring of the medical device should be transferred from the edge computing device to a vendor server; and
transfer the monitoring of the medical device from the edge computing device to the vendor server based on the analysis determining that the monitoring of the medical device should be transferred from the edge computing device to the vendor server.

9. The at least one non-transitory computer readable medium of claim 8, wherein the instructions further include:
transferring the monitoring of the medical device from the vendor server back to the edge computing device in response to the analysis no longer determining that the monitoring of the medical device should be transferred from the edge computing device to the vendor server.

10. The at least one non-transitory computer readable medium of claim 9, wherein the instructions further include
generating and transmitting a notification from the edge computing device to the vendor server, the notification being indicative of a readiness of the edge computing device to resume the monitoring.

11. The at least one non-transitory computer readable medium of claim 8, wherein the edge computing device is maintained by a customer of the medical device and configured to store and implement the one or more diagnostic models to analyze log data generated by the medical device.

12. The at least one non-transitory computer readable medium of claim 8, wherein the vendor server is maintained by a vendor of the medical device and configured to store a copy of the one or more diagnostic models.

13. The at least one non-transitory computer readable medium of claim 8, wherein the plurality of factors includes one or more of a status of connectivity of the edge computing device to the medical device, a presence of infrastructure at a site of the vendor server, and a number of technicians at the site of the edge computing device.

14. The at least one non-transitory computer readable medium of claim 13, wherein the plurality of factors further includes an availability of log data generated by the medical device and a presence of historical log data for the medical device.

15. The at least one non-transitory computer readable medium of claim 8, wherein the instructions further include:
generating an alert to be handled by a medical professional at a site of the edge computing device.

16. The at least one non-transitory computer readable medium of claim 15, wherein the instructions further include
determining an availability of medical professionals at the site of the edge computing device; and
if no medical professional is available, transferring the alert to the vendor server.

17. A distributed computing method, comprising:
monitoring a medical device by applying one or more diagnostic models to generate alerts indicative of problems with the medical device;
analyzing a plurality of factors to determine whether the monitoring of the medical device should be transferred from an edge computing device to a vendor server; and
generating an alert indicating that the monitoring of the medical device should be transferred from the edge computing device to the vendor server based on the analysis determining that the monitoring of the medical device should be transferred from the edge computing device to the vendor server.

18. The method of claim 17, further comprising:
transferring the monitoring of the medical device from the edge computing device to the vendor server based on the analysis determining that the monitoring of the medical device should be transferred from the edge computing device to the vendor server.

19. The method of claim 18, further comprising
transferring the monitoring of the medical device from the vendor server back to the edge computing device based on the analysis no longer determining that the monitoring of the medical device should be transferred from the edge computing device to the vendor server.

20. The method of claim 19, further comprising:
generating and transmitting a notification from the edge computing device to the vendor server, the notification being indicative of a readiness of the edge computing device to resume the monitoring.
